(19) United States
(12) Patent Application Publication
(10) Pub. No.: US 2025/0255859 A1
(43) Pub. Date: Aug. 14, 2025

(54) METHODS OF TREATING CFTR-MEDIATED DISEASES OR DISORDERS

(71) Applicant: Sionna Therapeutics Inc., Waltham, MA (US)

(72) Inventor: Gregory Donald Hurlbut, Boxborough, MA (US)

(21) Appl. No.: 19/193,542

(22) Filed: Apr. 29, 2025

Related U.S. Application Data
(63) Continuation of application No. PCT/US2023/036487, filed on Oct. 31, 2023.
(60) Provisional application No. 63/420,934, filed on Oct. 31, 2022.

(51) Int. Cl. A61K 31/47; A61K 31/353; A61K 31/404; A61K 31/4178; A61K 31/426; A61K 31/439; A61K 31/443; A61K 31/4439

(52) U.S. Cl. CPC A61K 31/47; A61K 31/353; A61K 31/404; A61K 31/4178; A61K 31/426; A61K 31/439; A61K 31/443; A61K 31/4439

(57) ABSTRACT

The present disclose includes, among other things, methods of treating or lessening the severity of CFTR-mediated disease or disorders.

### CROSS-REFERENCE TO RELATED APPLICATIONS

This application is a continuation of International Application No. PCT/US2023/036487, filed Oct. 31, 2023, which claims priority to U.S. Provisional Application No. 63/420,934, filed Oct. 31, 2022, the contents of both the international and provisional patent applications are incorporated herein by reference in their entireties.

### BACKGROUND

Cystic fibrosis (CF) results from CF transmembrane conductance regulator (CFTR) mutations, the most prevalent being ΔF508-CFTR. Approved CFTR modulators increase its function, providing eligible patients with clinical benefits. Despite advances, current modulators do not provide most people with CF with normal levels of CFTR function, indicated by the fact that, in most eligible patient groups, mean sweat chloride levels do not reach the normal range. ΔF508-CFTR results in loss of phenylalanine F508 within CFTR's first nucleotide binding domain (NBD1). ΔF508 causes NBD1 destabilization: a key driver of the impaired folding, trafficking, half-life, and function of ΔF508-CFTR. F508 also participates in the interface of NBD1 and the fourth intracellular loop (ICL4) of CFTR's second transmembrane domain (TMD). ΔF508weakens this interface, adding to ΔF508-CFTR's molecular pathology. There remains a need to find methods of treating CFTR-mediated diseases or disorders.

### SUMMARY

The present disclosure includes methods of treating CFTR-mediated diseases or disorders comprising administering a NBD1 corrector in combination with one or more additional therapeutic agents, including, but not limited to, a TMD1 corrector and a ICL4 corrector and/or a CFTR potentiator. As shown herein, and without being bound by any particular theory, full ΔF508-CFTR correction may require NBD1 stabilization, and without NBD1 stabilization, correction is significantly less. Compounds and methods described herein directly stabilize NBD1 and thus may improve patient health.

### BRIEF DESCRIPTION OF THE DRAWINGS

FIG. 1 depicts a graph showing ΔT aggregation (C) as compared to the ratio of ΔF508-NBD1 protein to compound. Compound 1 increased ΔF508-NBD1 stability by up to 10.4° C. Approved modulators elexacaftor (ELX), ivacaftor (IVA), tezacaftor (TEZ), and lumacaftor (LUM) had no meaningful impact on NBD1 stability.

FIG. 2 depicts a graph illustrating Compound 1 increases thermal stability of ΔF508-NBD1 and WT-NBD1. Compound 1-treated ΔF508-NBD1 has greater stability than that of untreated WT-NBD1.

FIG. 3 depicts a western blot and bar graph showing that Compound 1 improves ΔF508 trafficking. Western blot demonstrating that Compound 1 corrects ΔF508-CFTR to WT levels when used in combination with complementary modulators in CFSMEo− cells. TEZ binds to CFTR TMD1; based on our work, ELX acts to restore the ICL4 interface defect that results from F508del-CFTR and other CFTR mutations; ivacaftor is a CFTR potentiator with a negative impact on ΔF508-CFTR maturation. In all studies TEZ, ELX and IVA were used at their respective Eₘₐₓ concentrations.

FIG. 4 is a bar graph that shows Compound 1 increases cell surface ΔF508-CFTR to WT levels when combined with complementary modulators. An ΔF508-CFTR-horseradish peroxidase (HRP) trafficking assay was used to quantitate cell surface CFTR levels, following addition of a cell-impermeable HRP substrate to cells expressing ΔF508-CFTR or WT-CFTR with an HRP reporter inserted in frame into the CFTR 4th extracellular loop. Data represent the mean of 3 biological replicates, error bars display SE.

FIG. 5A is a bar graph that illustrates vehicle-subtracted forskolin (FSK) peak of 9 CFHBE donors treated as shown, compared with TEZ/IVA/ELX alone in the same donor, same experiment. Bars represent mean +/− SE of 6-8 replicates. CFTR-dependent chloride transport was measured in ΔF508 homozygous human bronchial epithelial cells (CFHBE) treated for 48 hours with Compound 1 plus TEZ/IVA/ELX at their respective Eₘₐₓ concentrations.

FIG. 5B is a bar graph that shows Mean data across donors, expressed as FSK response compared to the average FSK response (green bar) across a panel of 8 WT donors (6-8 replicates per, dotted lines are +/− SE). CFTR-dependent chloride transport was measured in ΔF508 homozygous human bronchial epithelial cells (CFHBE) treated for 48 hours with Compound 1 plus TEZ/IVA/ELX at their respective Eₘₐₓ concentrations.

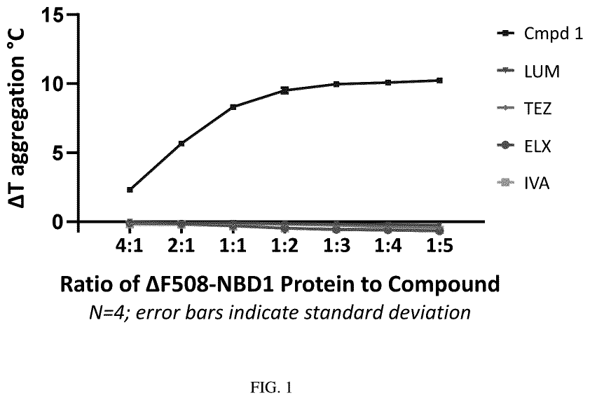

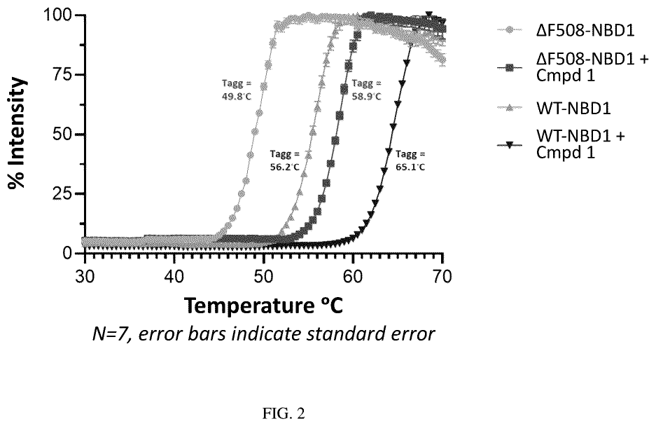

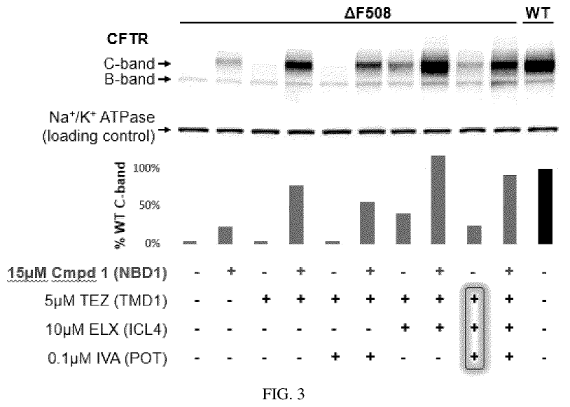

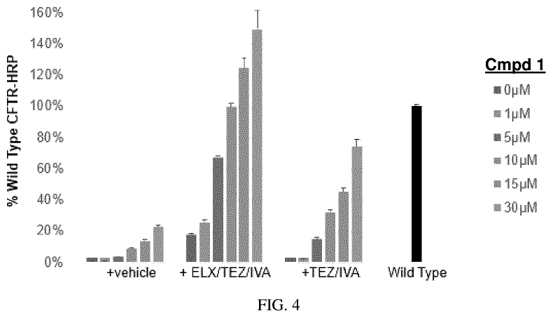

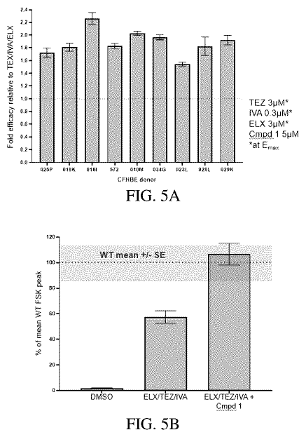

### DETAILED DESCRIPTION

### Definitions

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “subject”, as used herein, means an animal, preferably a mammal, and most preferably a human.

The term “pharmaceutically acceptable excipient” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

### Compounds of the Present Disclosure

In some embodiments, a compound of the present disclosure is a NBD1 corrector. In some embodiments, an NBD1 corrector is Compound 1:

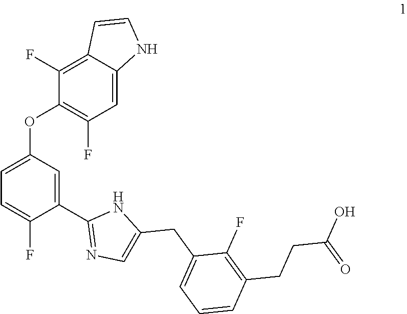 [SMILES: O=C(O)CCc1cccc(Cc2cnc(-c3cc(Oc4c(F)cc5[nH]ccc5c4F)ccc3F)[nH]2)c1F]

or a pharmaceutically acceptable salt thereof.

In some embodiments, a compound of the present disclosure is an ICL4 corrector. In some embodiments, an ICL4 corrector is elexacaftor (VX-445), vanzacaftor (VX-121) and Compound 2:

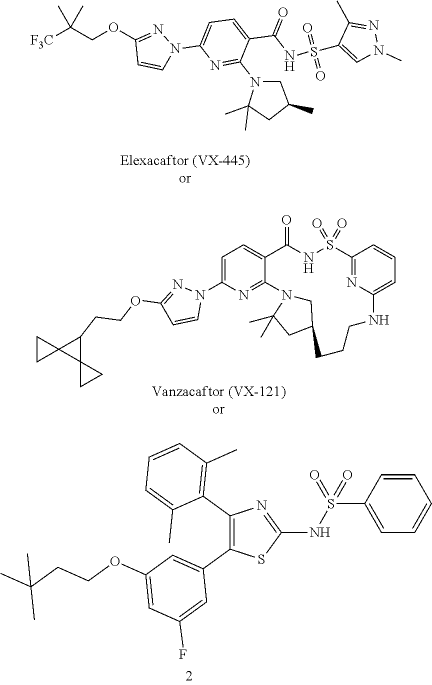

or a pharmaceutically acceptable salt thereof.

In some embodiments, a compound of the present disclosure is a TMD1 corrector. In some embodiments, the TMD1 corrector is a compound selected from the group consisting of lumacaftor (VX-809), tezacaftor (VX-661), galicaftor (ABBV-2222) and Compound 3:

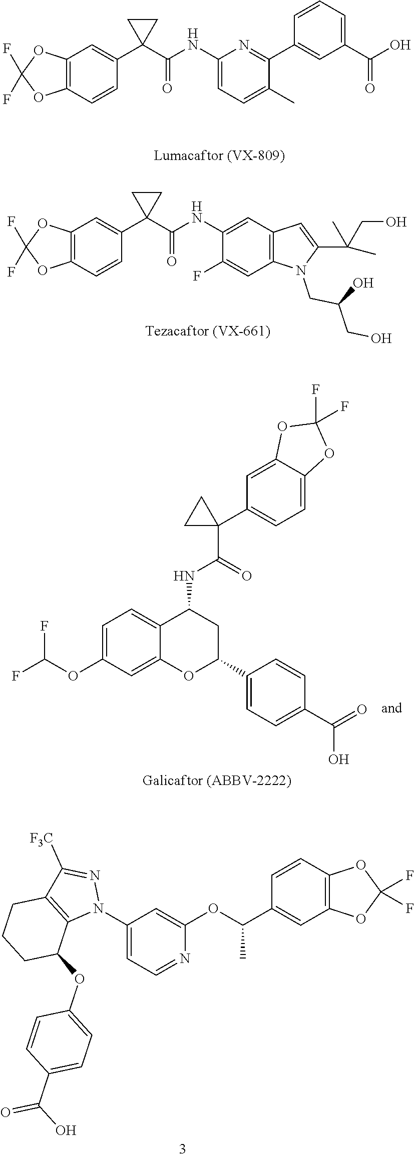 [SMILES: Cc1ccc(NC(=O)C2(c3ccc4c(c3)OC(F)(F)O4)CC2)nc1-c1cccc(C(=O)O)c1 ; CC(C)(CO)c1cc2cc(NC(=O)C3(c4ccc5c(c4)OC(F)(F)O5)CC3)c(F)cc2n1C[C@@H](O)CO ; O=C(O)c1ccc([C@H]2C[C@@H](NC(=O)C3(c4ccc5c(c4)OC(F)(F)O5)CC3)c3ccc(OC(F)F)cc3O2)cc1 ; C[C@H](Oc1cc(-n2nc(C(F)(F)F)c3c2[C@@H](Oc2ccc(C(=O)O)cc2)CCC3)ccn1)c1ccc2c(c1)OC(F)(F)O2]

or a pharmaceutically acceptable salt thereof.

In some embodiments, a compound of the present disclosure is a CFTR potentiator. In some embodiments, CFTR potentiator is:

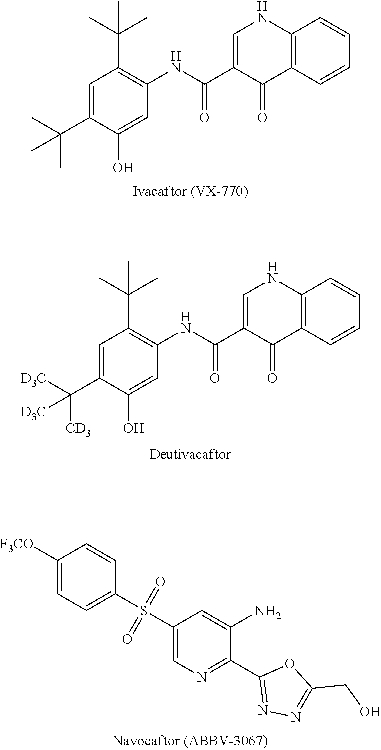 [SMILES: CC(C)(C)c1cc(C(C)(C)C)c(NC(=O)c2c[nH]c3ccccc3c2=O)cc1O ; [2H]C([2H])([2H])C(c1cc(C(C)(C)C)c(NC(=O)c2c[nH]c3ccccc3c2=O)cc1O)(C([2H])([2H])[2H])C([2H])([2H])[2H] ; Nc1cc(S(=O)(=O)c2ccc(OC(F)(F)F)cc2)cnc1-c1nnc(CO)o1]

or a pharmaceutically acceptable salt thereof.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound disclosed herein and a pharmaceutically acceptable carrier, adjuvant, or vehicle. In some embodiments, the amount of compound in compositions contemplated herein is such that is effective to measurably treat a disease or disorder in a biological sample or in a subject. In certain embodiments, the amount of compound in compositions of this disclosure is such that is effective to measurably treat a disease or disorder in a biological sample or in a subject. In certain embodiments, a composition contemplated by this disclosure is formulated for administration to a subject in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds disclosed herein may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds disclosed herein may be orally administered in any orally acceptable dosage form including, but not limited to, capsules, tablets, aqueous suspensions, or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring, or coloring agents may also be added.

Pharmaceutically acceptable compositions comprising a compound disclosed herein may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound disclosed herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

The presently disclosed compounds can be formulated into pharmaceutical compositions along with a pharmaceutically acceptable carrier or excipient. According to this aspect, there is provided a pharmaceutical composition comprising a compound disclosed herein in association with a pharmaceutically acceptable excipient, diluent or carrier.

The formulations of Compounds disclosed herein include those suitable for the administration routes detailed herein. They may conveniently be presented in unit dosage form and can be formulated in accordance with standard pharmaceutical practice as a pharmaceutical composition. Techniques and formulations generally and suitable for use herein are found in Remington's Pharmaceutical Sciences (16^{th} edition, Osol, A. Ed. (1980); Mack Publishing Co., Easton, PA). Such methods include the step of bringing into association the active ingredient with the excipient or carrier which constitutes one or more accessory ingredients. In general, the formulations are prepared by uniformly and intimately bringing into association the active ingredient with liquid excipients or carriers or finely divided solid excipients or carriers or both, and then, if necessary, shaping the product.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization of, for example, hydroxy methylcellulose or gelatin-microcapsules and poly-(methyl methacrylate) microcapsules, respectively, in colloidal drug delivery systems (for example, liposomes, albumin microspheres, microemulsions, nano-particles and nano capsules) or in macroemulsions. Such techniques are disclosed in Remington's Pharmaceutical Sciences 16^{th} edition, Osol, A. Ed. (1980).

In particular embodiments the pharmaceutical composition comprising the presently disclosed compounds further comprise a chemotherapeutic agent. In some of these embodiments, the chemotherapeutic agent is an immunotherapeutic agent.

### Methods of Using Compounds of the Present Disclosure

The present disclosure includes methods of treating CFTR-mediated diseases or disorders comprising administering a NBD1 corrector in combination with one or more additional therapeutic agents, including, but not limited to, a TMD1 corrector, a potentiator and an ICL4 corrector.

In some embodiments, the present disclosure includes method of treating CFTR-mediated disease or disorder in a subject in need thereof, comprising administering to the subject a therapeutically effective amount of an NBD1 corrector in combination with an ICL4 corrector. In some embodiments, an NBD1 corrector and an ICL4 corrector are administered simultaneously. In some embodiments, an NBD1 corrector is administered prior to an ICL4 corrector. In some embodiments a NBD1 corrector is administered after an ICL4 corrector.

In some embodiments, the present disclosure includes a method of treating CFTR-mediated disease or disorder in a subject in need thereof, comprising administered to the subject a therapeutically effective amount of an NBD1 corrector, wherein the subject has previously received treatment with an ICL4 corrector. In some embodiments, the present disclosure includes a method of treating CFTR-mediated disease or disorder in a subject in need thereof, comprising administered to the subject a therapeutically effective amount of an ICL4 corrector, wherein the subject has previously received treatment with an NBD1corrector.

In some embodiments, the present disclosure includes a method of treating CFTR-mediated disease or disorder in a subject in need thereof, comprising administering to the subject a therapeutically effective amount of compound 1 in combination with compound 2. In some embodiments, the present disclosure includes a method of treating CFTR-mediated disease or disorder in a subject in need thereof, comprising administering to the subject a therapeutically effective amount of compound 1 in combination with elexacaftor (ELX) or vanzacaftor.

In some embodiments, the present disclosure includes a method of treating CFTR-mediated disease or disorder in a subject in need thereof, comprising administering to the subject a therapeutically effective amount of an NBD1 corrector in combination with an ICL4 corrector and a TMD1 corrector. In some embodiments, the present disclosure includes a method of treating CFTR-mediated disease or disorder in a subject in need thereof, comprising administering to the subject a therapeutically effective amount of compound 1 in combination with compound 2 and compound 3. In some embodiments, the present disclosure includes a method of treating CFTR-mediated disease or disorder in a subject in need thereof, comprising administering to the subject a therapeutically effective amount of compound 1 in combination with elexacaftor or vanzacaftor and compound 3. In some embodiments, the present disclosure includes a method of treating CFTR-mediated disease or disorder in a subject in need thereof, comprising administering to the subject a therapeutically effective amount of compound 1 in combination with compound 2 and tezacaftor (TEZ) or lumacaftor (LUM) or galicaftor. In some embodiments, the present disclosure includes a method of treating CFTR-mediated disease or disorder in a subject in need thereof, comprising administering to the subject a therapeutically effective amount of compound 1 in combination with elexacaftor (ELX) or vanzacaftor and tezacaftor (TEZ) or lumacaftor (LUM) or galicaftor.

In some embodiments, the present disclosure includes a method of treating CFTR-mediated disease or disorder in a subject in need thereof, comprising administering to the subject a therapeutically effective amount of an NBD1 corrector in combination with an ICL4 corrector, a TMD1 corrector, and a CFTR potentiator. In some embodiments, the present disclosure includes a method of treating CFTR-mediated disease or disorder in a subject in need thereof, comprising administering to the subject a therapeutically effective amount of compound 1 in combination with elexacaftor (ELX) or vanzacaftor, tezacaftor (TEZ) or lumacaftor (LUM) or galicaftor, and ivacaftor (IVA) or deutivacaftor or navocaftor. In some embodiments, the present disclosure includes a method of treating CFTR-mediated disease or disorder in a subject in need thereof, comprising administering to the subject a therapeutically effective amount of compound 1 in combination with a compound 2, tezacaftor (TEZ) or lumacaftor (LUM) or galicaftor, and ivacaftor (IVA) or navocaftor or deutivacaftor.

In some embodiments, the present disclosure includes a method of treating CFTR-mediated disease or disorder in a subject in need thereof, comprising administering to the subject a therapeutically effective amount of an NBD1 corrector in combination with Trikafta. In some embodiments, the present disclosure includes a method of treating CFTR-mediated disease or disorder in a subject in need thereof, comprising administering to the subject a therapeutically effective amount of compound 1 in combination with Trikafta.

In some embodiments, the present disclosure includes a method of treating CFTR-mediated disease or disorder in a subject in need thereof, comprising administering to the subject a therapeutically effective amount of an NBD1 corrector in combination with an TMD1 corrector. In some embodiments, the present disclosure includes a method of treating CFTR-mediated disease or disorder in a subject in need thereof, comprising administering to the subject a therapeutically effective amount of an NBD1 corrector in combination with compound 3.

### EXEMPLIFICATION

### Analytical Procedures

High Pressure Liquid Chromatography-Mass Spectrometry (LC-MS) to determine compound retention times (RT) and associated mass ions were performed using one of the following methods.

LC-MS Method 1: Mobile Phase: A: water (0.01% TFA). B: ACN (0.01% TFA). Gradient: 5%-95% B in 1.5 min. Flow Rate: 2.0 mL/min. Column: Sunfire C18, 4.6×50 mm, 3.5 μm. Oven Temperature: 50° C. Mass Range: 110-1000. UV (214 nm, 254 nm).

LC-MS Method 2: Column: Xbridge C18(2) (4.6×50 mm, 3.5 μm). Mobile phase: H₂O (10 mmol NH₄HCO₃) (A)/ACN (B). Elution program: Gradient from 10 to 95% of B in 1.5 min at 1.8 mL/min. Temperature: 50° C. Detection: UV (214 nm, 254 nm) and MS (ESI, Positive mode, 103 to 800 amu).

The ¹H NMR spectra were collected at 400 MHz on a Gemini 400 or Varian Mercury 400 spectrometer (unless noted otherwise) with an ASW 5 mm probe, and usually recorded at ambient temperature in a deuterated solvent, such as D₂O, DMSO-D₆, CH₃OH-d₄ or CDCl₃ unless otherwise noted. Chemical shift values (δ) are indicated in parts per million (ppm) with reference to tetramethylsilane (TMS) as the internal standard.

### Abbreviations

ACN: acetonitrile
Boc: tert-butyloxycarbonyl
DEA: diethyl amine
DBU: 1,8-Diazabicyclo[5.4.0]undec-7-ene
DCE: 1,2-dichloroethane
DCM: dichloromethane
DIAD: diisopropyl azodicarboxylate
DMAP: 4-dimethylaminopyridine
DMSO: dimethyl sulfoxide
dppf: 1,1′-Bis(diphenylphosphino)ferrocene
EA: ethyl acetate
ee: enantiomeric excess
ESI: electron spray ionization
HPLC: high performance liquid chromatography
LC-MS: liquid chromatography-mass spectrometry
MsCl: methanesulfonyl chloride
Pd/C: Palladium on carbon
rt: room temperature
PE: petroleum ether
SFC: supercritical fluid chromatography
TBS: tert-butyldimethylsilyl
TIPS: triisopropylsilyl
THF: tetrahydrofuran
THP: tetrahydropyran
Ts: tosyl

### GENERAL DISCLAIMER ABOUT STEREOCHEMISTRY

It is understood that absolute stereochemistries for all intermediates and examples described herein have not been determined. The assignments of the chiral center(s) to R or S are completely arbitrary and are solely for the purpose of differentiating the different fractions (P1 and P2) eluted out from either flash column chromatography or prep-HPLC, or chiral HPLC, or SFC. There is no association of P1 or P2 with the S or R designations.

Both ChemDraw and Mol2Nam from OpenEye Scientific software have been used to generate compounds' names. If not consistent, the structure should govern.

### Example 1. Synthesis of Compound 1: 3-(3-((2-(5-((4,6-Difluoro-1H-indol-5-yl)oxy)-2-fluorophenyl)-1H-imidazol-5-yl)methyl)-2-fluorophenyl)propanoic Acid

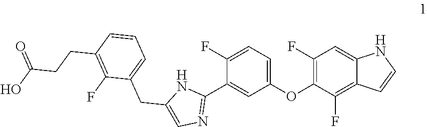 [SMILES: O=C(O)CCc1cccc(Cc2cnc(-c3cc(Oc4c(F)cc5[nH]ccc5c4F)ccc3F)[nH]2)c1F]

A. 2-(3-(3-Ethoxy-3-oxoprop-1-en-1-yl)-2-fluorophenyl)acetic Acid

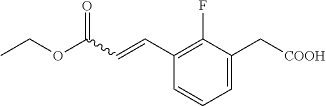 [SMILES: CCOC(=O)C=Cc1cccc(CC(=O)O)c1F]

To a stirred solution of 2-(3-bromo-2-fluorophenyl)acetic acid (4.62 g, 19.8 mmol) in DMF (100 mL) was added ethyl acrylate (4.4 mL, 40 mmol), triethylamine (14.0 mL, 100 mmol), tri(o-tolyl)phosphine (1.41 g, 4.63 mmol) and Pd(OAc)₂ (0.450 g, 2.00 mmol). The reaction was heated overnight at 120° C., cooled to room temperature and diluted with 1.0 N hydrochloric acid. This mixture was extracted with ethyl acetate (3×100 mL). The combined extracts were washed with water (3×200 mL) and brine (1×200 mL), dried over sodium sulfate and concentrated. The residue was purified by flash chromatography over silica (50% ethyl acetate in petroleum ether) to afford the title compound as a white solid (3.50 g, 70%). MS: 253 m/z [M+H]⁺.

B. 2-(3-(3-Ethoxy-3-oxopropyl)-2-fluorophenyl)acetic Acid

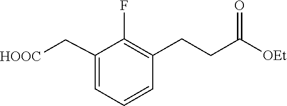 [SMILES: CCOC(=O)CCc1cccc(CC(=O)O)c1F]

A stirred suspension of step A product (3.50 g, 13.9 mmol) and 10% Pd/C (1.00 g) in ethanol (100 mL) was cycled between vacuum and a nitrogen atmosphere three times. The reaction vessel was evacuated a final time and backfilled with hydrogen (via balloon). The reaction was allowed to proceed overnight and then filtered through a pad of Celite, which was subsequently rinsed with ethanol (˜200 mL). The combined filtrate was concentrated, and the residue was purified by flash chromatography over silica (50-70% ethyl acetate in hexane) to afford the title compound as a solid (2.20 g, 62%). MS: 255 m/z [M+H]⁺.

C. Ethyl 3-(3-(3-chloro-2-oxopropyl)-2-fluorophenyl)propanoate

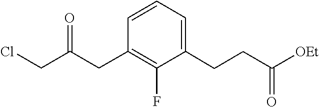 [SMILES: CCOC(=O)CCc1cccc(CC(=O)CCl)c1F]

To a stirred and cooled (0° C.) solution of step B product (1.01 g, 3.97 mmol) in dichloromethane (20 mL) was added oxalyl chloride (0.41 mL, 4.8 mmol) followed by two drops of DMF. The reaction was warmed to room temperature, stirred for two hours and concentrated. The resulting oil was co-evaporated several times with heptane (20 mL) to remove any trace oxalyl chloride. The crude acid chloride was dissolved in 1:1 acetonitrile/THF (20 mL). To this stirred and cooled (0° C.) solution was added, dropwise over five minutes, a 2.0 M solution of trimethylsilyldiazomethane in diethyl ether (8.0 mL, 16 mmol). The reaction was warmed to room temperature, stirred overnight and then concentrated. The crude α-diazoketone was dissolved in dichloromethane (15 mL). To this stirred and cooled (0° C.) solution was added, dropwise over five minutes, a 4.0 M solution of hydrogen chloride in 1,4-dioxane (4.0 mL, 16 mmol; vigorous gas evolution was observed). The reaction was warmed to room temperature, stirred for an additional one hour and then diluted with water (50 mL). This mixture was extracted with ethyl acetate (2×50 mL). The combined extracts were washed with brine (1×50 mL), dried over sodium sulfate and concentrated. The residue was purified by flash chromatography over silica (10-25% ethyl acetate in hexane) to afford the title compound as an oil (0.515 g, 45% over three steps). ¹H NMR (400 MHz, CDCl₃)δ7.16 (dt, J=13.2, 5.7 Hz, 1H), 7.09-6.99 (m, 2H), 4.18 (s, 2H), 4.12 (q, J=7.2 Hz, 2H), 3.91 (s, 2H), 2.98 (t, J=7.6 Hz, 2H), 2.61 (t, J=7.6 Hz, 2H), 1.23 (t, J=7.2 Hz, 3H) ppm. MS: 287 m/z [M+H]⁺.

D. 5-(2,6-Difluoro-4-nitrophenoxy)-2-fluorobenzonitrile

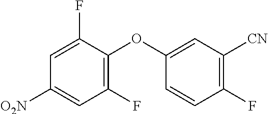 [SMILES: N#Cc1cc(Oc2c(F)cc([N+](=O)[O-])cc2F)ccc1F]

To a stirred solution of 1,2,3-trifluoro-5-nitrobenzene (1.10 g, 6.21 mmol) in DMF (5 mL) was added potassium carbonate (1.71 g, 12.4 mmol) and 2-fluoro-5-hydroxybenzonitrile (0.936 g, 6.83 mmol). The reaction was heated at 100° C. for two hours and then cooled to room temperature and diluted with water (20 mL). The precipitate which formed was collected by suction filtration, washed with water and vacuum oven dried to afford the title compound as a light yellow solid (1.70 g, 94%). ¹H NMR (400 MHZ, CDCl₃)δ8.06-8.01 (m, 2H), 7.30-7.20 (m, 3H) ppm.

E. 5-(4-Amino-2,6-difluorophenoxy)-2-fluorobenzonitrile

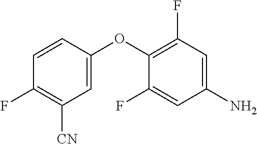 [SMILES: N#Cc1cc(Oc2c(F)cc(N)cc2F)ccc1F]

To a stirred suspension of step D product (1.70 g, 5.78 mmol) in ethanol (30 mL) was added a solution of ammonium chloride (2.45 g, 46.2 mmol) in water (10 mL) followed by iron power (1.48 g, 23.1 mmol). The reaction was heated at reflux for four hours and then cooled to room temperature and filtered to remove the solids. The filter cake was rinsed with additional ethanol (˜30 mL) and the combined filtrate was concentrated. The residue was dissolved in ethyl acetate (100 mL) and this solution was washed with water (3×30 mL) and brine (1×30 mL), dried over sodium sulfate and concentrated. Crude title compound, which was used without purification, was afforded as a yellow solid (1.50 g, 98%). MS: 263 m/z [M-H]⁻.

F. 5-(4-Amino-2,6-difluoro-3-iodophenoxy)-2-fluorobenzonitrile

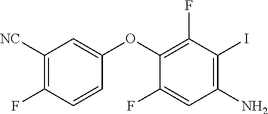 [SMILES: N#Cc1cc(Oc2c(F)cc(N)c(I)c2F)ccc1F]

To a stirred solution of step E product (1.50 g, 5.68 mmol) in acetic acid (12 mL) was added NIS (1.36 g, 6.04 mmol). The reaction was heated at 30° C. for one hour and then concentrated. The residue was purified by flash chromatography over silica (20% ethyl acetate in petroleum ether) to afford the title compound as a yellow solid (1.93 g, 87%). MS: 389 m/z [M-H]⁻.

G. 5-(4-Amino-2,6-difluoro-3-((trimethylsilyl)ethynyl)phenoxy)-2-fluorobenzonitrile

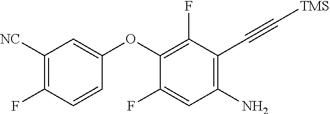 [SMILES: C[Si](C)(C)C#Cc1c(N)cc(F)c(Oc2ccc(F)c(C#N)c2)c1F]

To a stirred solution of step F product (1.00 g, 2.56 mmol) in DMF (15 mL) was added triethylamine (0.54 mL, 3.84 mmol), ethynyltrimethylsilane (0.47 mL, 3.39 mmol), Pd(dppf)Cl₂ (0.182 mg, 0.249 mmol) and CuI (48.0 mg, 0.252 mmol). The reaction was heated at 30° C. for three hours and then partitioned between water (30 mL) and ethyl acetate (30 mL). The organic layer was combined with additional extracts (ethyl acetate, 2×20 mL), washed with brine (1×10 mL) and dried with sodium sulfate. The solution was concentrated and the residue was purified by flash chromatography over silica (20% ethyl acetate in petroleum ether) to afford the title compound as a yellow solid (0.900 g, 98%). MS: 359 m/z [M-H]⁻.

H. 5-((4,6-Difluoro-1H-indol-5-yl)oxy)-2-fluorobenzonitrile

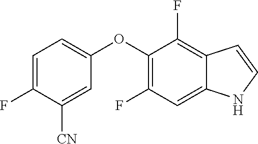 [SMILES: N#Cc1cc(Oc2c(F)cc3[nH]ccc3c2F)ccc1F]

To a stirred solution of step G product (0.900 g, 2.50 mmol) in DMF (5 mL) was added CuI (0.952 g, 5.00 mmol). The reaction was heated overnight at 100° C. After this time, the mixture was cooled to room temperature and filtered free of solids. The filtrate was diluted with ethyl acetate (100 mL) and the solution was washed with brine (1×50 mL), dried over sodium sulfate and concentrated. The residue was purified by flash chromatography over silica (20% ethyl acetate in petroleum ether) to afford the title compound as a yellow solid (0.440 g, 61%). ¹H NMR (400 MHZ, DMSO-d₆)δ11.64 (s, 1H), 7.63-7.61 (m, 1H), 7.51-7.47 (m, 2H), 7.39-7.35 (m, 1H), 7.32 (d, J=10.4 Hz, 1H), 6.58-6.57 (m, 1H) ppm. MS: 287 m/z [M-H]⁻.

I. 5-((4,6-Difluoro-1H-indol-5-yl)oxy)-2-fluorobenzimidamide

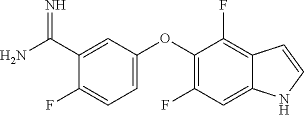 [SMILES: N=C(N)c1cc(Oc2c(F)cc3[nH]ccc3c2F)ccc1F]

To a stirred and cooled (° C.) solution of step H product (0.440 g, 1.53 mmol) in THF (10 mL) was added a 1.0 M solution of lithium bis(trimethylsilyl) amide in THF (6.1 mL, 6.1 mmol). The reaction was allowed to warm to room temperature and stirred overnight. Following this period, the reaction was quenched with addition of saturated aqueous ammonium chloride solution (30 mL) and then extracted with ethyl acetate (2×30 mL). The combined extracts were washed with brine (2×30 mL), dried with sodium sulfate and concentrated. Crude title compound, which was used without purification, was afforded as a light amber solid (0.500 g, 107%). MS: 306 m/z [M+H]⁺.

J. Ethyl 3-(3-((2-(5-((4,6-difluoro-1H-indol-5-yl)oxy)-2-fluorophenyl)-1H-imidazol-5-yl)methyl)-2-fluorophenyl)propanoate

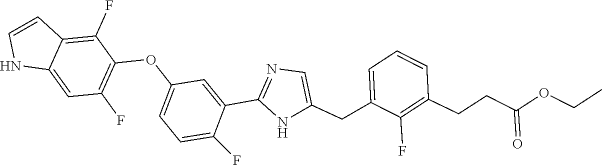 [SMILES: CCOC(=O)CCc1cccc(Cc2cnc(-c3cc(Oc4c(F)cc5[nH]ccc5c4F)ccc3F)[nH]2)c1F]

A stirred solution of step I product (0.100 g, 0.328 mmol), step C product (0.085 g, 0.296 mmol), and sodium bicarbonate (50.0 mg, 0.596 mmol) in DMF (2 mL) was heated overnight at 70° C. The reaction mixture was then cooled to room temperature, diluted with water (50 mL) and extracted with ethyl acetate (3×40 mL). The combined organic extracts were washed with aqueous lithium chloride and aqueous sodium chloride solutions (each, 1×50 mL), dried over sodium sulfate and concentrated. The residue was purified by flash chromatography over silica (50% ethyl acetate in petroleum ether) to afford the title compound as pale yellow oil (0.120 g, 75%). MS: 538 m/z [M+H]⁺.

K. Compound 1, 3-(3-((2-(5-((4,6-Difluoro-1H-indol-5-yl oxy)-2-fluorophenyl)-1H-imidazol-5-yl) methyl)-2-fluorophenyl)propanoic Acid

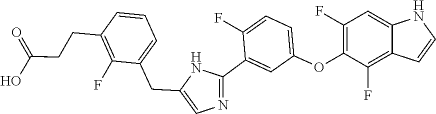 [SMILES: O=C(O)CCc1cccc(Cc2cnc(-c3cc(Oc4c(F)cc5[nH]ccc5c4F)ccc3F)[nH]2)c1F]

To a stirred solution of step J product (2.40 g, 5.20 mmol) in a mixture of THF (39 mL), methanol (13 mL) and water (13 mL) was added lithium hydroxide monohydrate (1.09 g, 26.0 mmol). After two hours at room temperature the mixture was made acidic (˜pH 4) by the addition of 1.0 N hydrochloric acid. The resulting suspension was partitioned between ethyl acetate (100 mL) and brine (1×30 mL). The organic layer was dried over sodium sulfate and concentrated to afford the title compound as white solid (2.40 g, 91%). ¹H NMR (400 MHz, CD₃OD)δ7.39 (dd, J=6.0, 3.2 Hz, 1H), 7.19 (d, J=3.2 Hz, 1H), 7.12-6.95 (m, 4H), 6.92-6.81 (m, 2H), 6.65 (s, 1H), 6.44 (d, J=2.6 Hz, 1H), 3.86 (s, 2H), 2.84 (t, J=7.6 Hz, 2H), 2.48 (t, J=7.6 Hz, 2H) ppm. MS: 510 m/z [M+H]⁺.

### Example 2. Synthesis of Compound 2: N-(5-(3-(3,3-Dimethylbutoxy)-5-fluorophenyl)-4-(2,6-dimethylphenyl)thiazol-2-yl)benzenesulfonamide

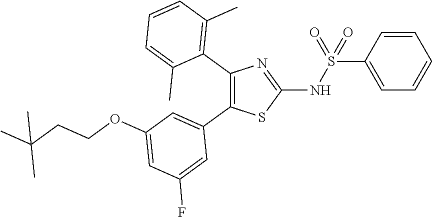 [SMILES: Cc1cccc(C)c1-c1nc(NS(=O)(=O)c2ccccc2)sc1-c1cc(F)cc(OCCC(C)(C)C)c1]

### Step 1.

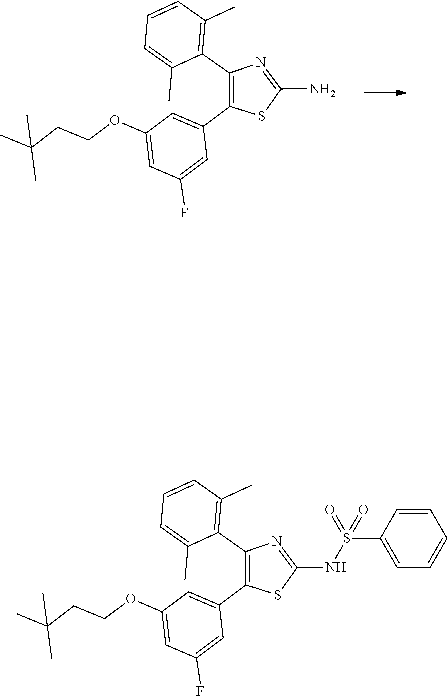 [SMILES: Cc1cccc(C)c1-c1nc(N)sc1-c1cc(F)cc(OCCC(C)(C)C)c1 ; Cc1cccc(C)c1-c1nc(NS(=O)(=O)c2ccccc2)sc1-c1cc(F)cc(OCCC(C)(C)C)c1]

The mixture of 5-[3-(3,3-dimethylbutoxy)-5-fluoro-phenyl]-4-(2,6-dimethylphenyl)thiazol-2-amine (Intermediate C-6a as described in WO2021/097057) (300 mg, 0.75 mmol) in pyridine (3.0 mL) was added benzenesulfonyl chloride (0.192 mL, 1.51 mmol). The reaction was stirred at 130° C. in a microwave oven for 3 h. The reaction was cooled to rt and then diluted with brine (20 mL). The aqueous solution was extracted with ethyl acetate (40 mL×2). The combined organics were dried over anhydrous sodium sulfate, filtered, and concentrated. The residue was purified by Prep-HPLC to afford the title compound, N-[5-[3-(3,3-dimethylbutoxy)-5-fluoro-phenyl]-4-(2,6-dimethylphenyl)thiazol-2-yl]benzenesulfonamide (206 mg, 51%) as a white solid. LCMS: LC retention time 1.76 min. MS (ESI) m/z 539 [M+H]⁺. ¹H NMR (400 MHz, chloroform-d)δ9.73 (s, 1H), 7.91 (d, J=7.5 Hz, 2H), 7.52 (dt, J=32.1, 7.3 Hz, 3H), 7.35-7.04 (m, 3H), 6.55-6.21 (m, 3H), 3.66 (t, J=7.2 Hz, 2H), 2.13 (s, 6H), 1.61 (t, J=7.1 Hz, 2H), 0.95 (s, 9H) ppm.

### Example 3. Compound 3. Synthesis of 3-4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol- 7-yl]oxy]benzoic Acid

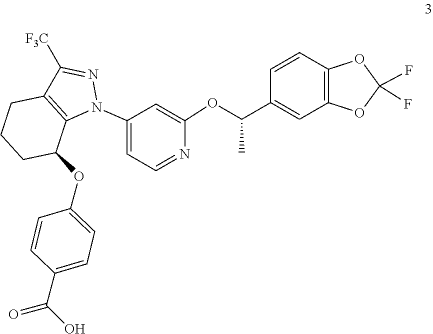 [SMILES: C[C@H](Oc1cc(-n2nc(C(F)(F)F)c3c2[C@@H](Oc2ccc(C(=O)O)cc2)CCC3)ccn1)c1ccc2c(c1)OC(F)(F)O2]

### Synthetic Method 1 of the Synthesis of Example 3, Compound 3

Step 1. Synthesis of 4-bromo-2-(1-phenylethoxy)pyridine

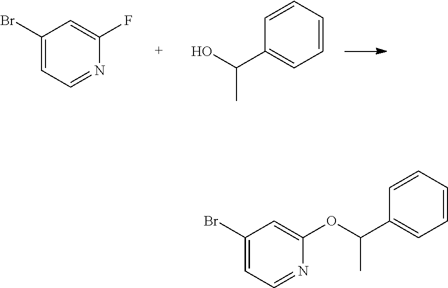

To the solution of 4-bromo-2-fluoro-pyridine (20.0 g, 114 mmol) and 1-phenylethanol (13.9 g, 114 mmol) in DMF (200 mL) was added cesium carbonate (111081 mg, 341 mmol). Then the mixture was stirred at 100° C. overnight. The crude product was then purified by flash chromatography eluted with 10% EtOAc in isohexane to get the title compound (29.0 g, 92% yield) as a yellow solid. LC-MS (Method 1): Retention time=2.45 min. MS (ESI) m/z 174.1/176.1 (M-PhCHMe+H)⁺.

Step 2. Synthesis of Tert-butyl 1-(2-(1-phenylethoxy)pyridin-4-yl)hydrazinecarboxylate

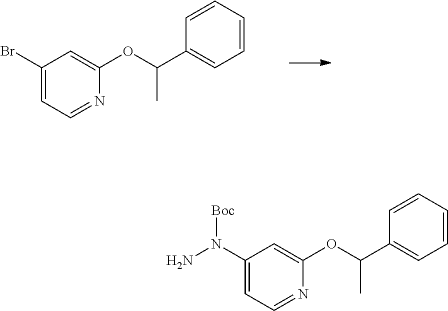 [SMILES: CC(Oc1cc(Br)ccn1)c1ccccc1 ; CC(Oc1cc(N(N)C(=O)OC(C)(C)C)ccn1)c1ccccc1]

To a solution of 4-bromo-2-(1-phenylethoxy)pyridine (15.0 g, 53.9 mmol) in 1,4-dioxane (150 mL) was added tert-butyl N-aminocarbamate (8.5 g, 64.7 mmol), (9,9-dimethyl-9H-xanthene-4,5-diyl)bis(diphenylphosphine) (3.1 mg, 5.39 mmol), tris(dibenzylideneacetone) dipalladium (4.9 g, 5.39 mmol), and cesium carbonate (52.7 g, 162 mmol). Then the mixture was stirred at 100° C. overnight under Ar. The crude product was then purified by flash column chromatography eluted with 10% EtOAc in isohexane to get the title compound (16.0 g, 90% yield) as a yellow solid. LC-MS (Method 1): Retention time=2.11 min. MS (ESI) m/z 330 (M+H)⁺.

Step 3. Synthesis of 1-(2-hydroxypyridin-4-yl)-3-(trifluoromethyl)-1,4,5,6-tetrahydro-7H-indazol-7-one

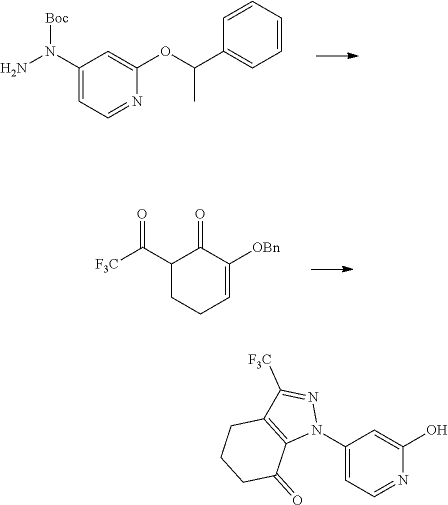 [SMILES: CC(Oc1cc(N(N)C(=O)OC(C)(C)C)ccn1)c1ccccc1 ; O=C1C(OCc2ccccc2)=CCCC1C(=O)C(F)(F)F ; O=C1CCCc2c(C(F)(F)F)nn(-c3ccnc(O)c3)c21]

To a solution of tert-butyl 1-(2-(1-phenylethoxy)pyridin-4-yl)hydrazinecarboxylate (10 g, 30.4 mmol) and 2-(benzyloxy)-6-(2,2,2-trifluoroacetyl)cyclohex-2-en-1-one in 2,2,2-trifluoroethanol (100 mL) was added sulfuric acid (25 mL). Then the mixture was stirred at 80° C. overnight. The mixture was poured into water (500 mL) and the pH was adjusted to 8-9 with NaHCO₃, and then extracted with EA (3×50 mL). The organic layers were combined, washed with brine, dried over Na₂SO₄, filtered and concentrated. The crude product was then purified by flash column chromatography eluted with 10% EtOAc in isohexane to get the title compound (5 g, 55% yield) as a yellow solid. LC-MS (Method 1): Retention time=1.72 min. MS (ESI) m/z 298.3 (M+H)⁺

Step 4. Synthesis of 1-(2,2-difluorobenzo[d][1,3]dioxol-5-yl)ethan-1-one

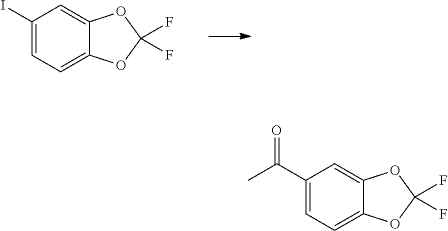

To a solution of 1-ethoxyvinyltri-n-butyltin (1.7 mL, 4.93 mmol) in toluene (20 mL) was added 2,2-difluoro-5-iodo-1,3-benzodioxole (1.40 g, 4.93 mmol) and Pd(PPh₃)₂Cl₂ (138 mg, 0.20 mmol). The mixture was stirred at 90° C. for 12 h under N₂, then cooled to rt. HCl (30 mL, 60.0 mmol) was added and stirred for 0.5 h. The mixture was extracted with EA (30 mL×3). The organic layers were combined, washed with brine (30 mL), dried over Na₂SO₄, filtered and concentrated. The crude product was purified by flash column chromatography eluted with 5% EtOAc in isohexane to get the title compound (950 mg, 95% yield) as a colorless oil. LC-MS (Method 1): Retention time=2.02 min. MS (ESI) m/z 201.0 (M+H)⁺. ¹H NMR (500 MHz, CDCl₃)δ7.77 (dd, J=8.3, 1.6 Hz, 1H), 7.69 (d, J=1.6 Hz, 1H), 7.14 (d, J=8.3 Hz, 1H), 2.60 (s, 3H).

Step 5. Synthesis of 1-(2,2-difluorobenzo[d][1,3]dioxol-5-yl)ethan-1-ol

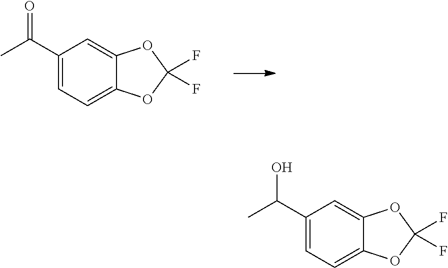

To a solution of 1-(2,2-difluoro-1,3-benzodioxol-5-yl)ethanone (900 mg, 4.50 mmol) in methanol (10 mL) was added NaBH₄ (340 mg, 8.99 mmol) at 0° C. and stirred for 2 h. Then the mixture was poured into water (40 mL) and extracted with EA (3×30 mL). The organic layers were combined, washed with brine (30 mL), dried over Na₂SO₄, filtered and concentrated. The crude was purified by flash column chromatography eluted with 10% EtOAc in isohexane to get the title compound (860 mg, 94.6% yield) as a colorless oil. LC-MS (Method 1): Retention time=1.94 min. MS (ESI) m/z 185.0 (M-H₂O+H)⁺.

Step 6. Synthesis of 1-(2-(1-(2,2-difluorobenzo[d][1,3]dioxol-5-yl)ethoxy)pyridin-4-yl)-3-(trifluoromethyl)-1,4,5,6-tetrahydro-7H-indazol- 7-one

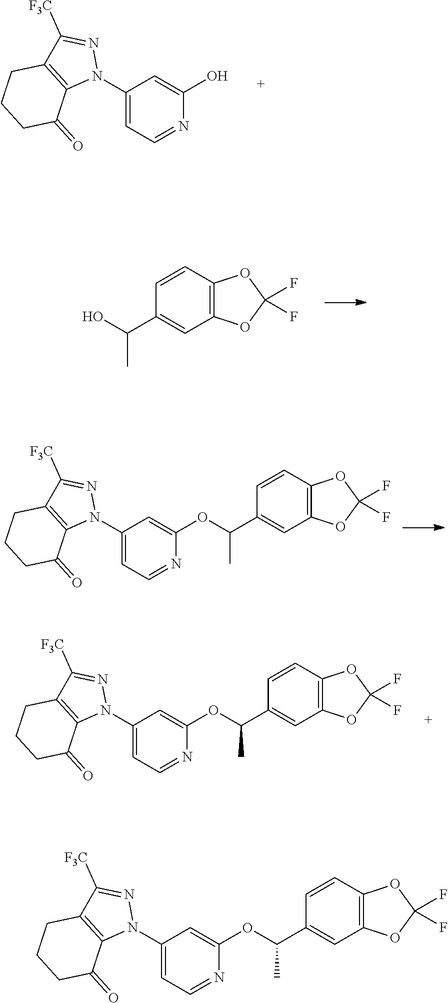 [SMILES: O=C1CCCc2c(C(F)(F)F)nn(-c3ccnc(O)c3)c21 ; CC(O)c1ccc2c(c1)OC(F)(F)O2 ; CC(Oc1cc(-n2nc(C(F)(F)F)c3c2C(=O)CCC3)ccn1)c1ccc2c(c1)OC(F)(F)O2 ; C[C@@H](Oc1cc(-n2nc(C(F)(F)F)c3c2C(=O)CCC3)ccn1)c1ccc2c(c1)OC(F)(F)O2 ; C[C@H](Oc1cc(-n2nc(C(F)(F)F)c3c2C(=O)CCC3)ccn1)c1ccc2c(c1)OC(F)(F)O2]

To a solution of 1-(2-hydroxy-4-pyridyl)-3-(trifluoromethyl)-5,6-dihydro-4H-indazol-7-one (4000 mg, 13.5 mmol) in THF (50 mL) were added 1-(2,2-difluoro-1,3-benzodioxol-5-yl)ethanol (3265 mg, 16.1 mmol), and triphenylphosphine (4236 mg, 16.1 mmol). After the solution was cooled to 0° C., diethyl azodicarboxylate (2.6 mL, 16.1 mmol) was added dropwise at 0° C. and then stirred at 25° C. for 1 h. The solution was concentrated, and the crude product was then purified by flash column chromatography eluted with 10% EtOAc in isohexane to obtain the racemic product (4500 mg, 69% yield) which was purified by SFC to obtain both enantiomers of the title compound. The enantiomer eluting first (more mobile fractions) was designated as P1 (2100 mg, 47% yield) and the enantiomer eluting second designated as P2 (2000 mg, 44% yield), respectively. LC-MS (Method 2): Retention time=2.36 min. MS (ESI) m/z 482.0 (M+H)⁺.

Step 7. Synthesis of 1-(2-((S)-1-(2,2-difluorobenzo[d][1,3]dioxol-5-yl)ethoxy)pyridin-4-yl)-3-(trifluoromethyl)-4,5,6,7-tetrahydro-1H- indazol-7-ol

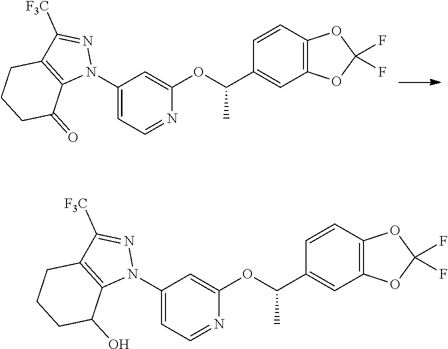 [SMILES: C[C@H](Oc1cc(-n2nc(C(F)(F)F)c3c2C(=O)CCC3)ccn1)c1ccc2c(c1)OC(F)(F)O2 ; C[C@H](Oc1cc(-n2nc(C(F)(F)F)c3c2C(O)CCC3)ccn1)c1ccc2c(c1)OC(F)(F)O2]

To a solution of 1-[2-[(1R)-1-(2,2-difluoro-1,3-benzodioxol-5-yl)ethoxy]-4-pyridyl]-3-(trifluoromethyl)-5,6-dihydro-4H-indazol-7-one (P2 of Step 6 above) (400 mg, 0.831 mmol) in methanol (10 mL) was added sodium borohydride (126 mg, 3.32 mmol). Then the mixture was stirred at rt for 2 h under argon. Then the mixture was quenched with water (50 mL) and extracted with EA (50 mL×3). The organic layer was washed with brine (20 mL) and dried over Na₂SO₄, filtered and concentrated. The crude was purified by flash chromatography (Biotage, 40 g silica gel column @ 60 mL/min, eluting with 0-30% ethyl acetate in petroleum for 20 min) to get the title compound (350 mg, 82.8%) as a light yellow oil. LCMS (Method 1): Rt 2.38 min. MS (ESI) m/z 300.1 (M+H)⁺.

Step 8. Synthesis of Methyl 4-((1-(2-((R)-1-(2,2-difluorobenzo[d][1,3]dioxol-5-yl)ethoxy)pyridin-4-yl)-3-(trifluoromethyl)-4,5,6,7- tetrahydro-1H-indazol-7-yl)oxy)benzoate

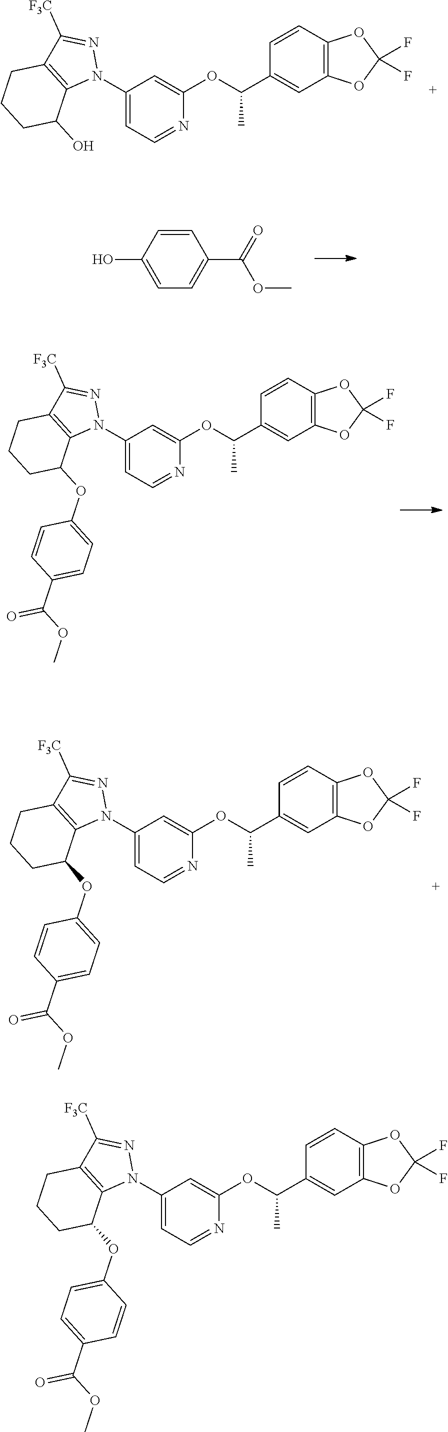

To the solution of 1-[2-[(1S)-1-(2,2-difluoro-1,3-benzodioxol-5-yl)ethoxy]-4-pyridyl]-3-(trifluoromethyl)-4,5,6,7-tetrahydroindazol-7-ol (product from Step 7 above) (800 mg, 1.65 mmol) in THF (10 mL) were added methyl 4-hydroxybenzoate (302 mg, 1.99 mmol) and PPh₃ (521 mg, 1.99 mmol). After the solution was stirred at 0° C. for 5 min, DEAD (0.31 mL, 1.99 mmol) was added dropwise at 0° C. The solution was then stirred at 25° C. for 1 h. The solution was concentrated, and the crude product was then purified by flash column chromatography eluted with 10% ethyl acetate in isohexane to get the mixture of diastercomers (650 mg, 69% yield) which was further separated by SFC to get the title compounds with the first fraction designated as P1 (300 mg, 47%; a yellow oil), and the second fractions as P2 (300 mg, 44%; a yellow oil). LC-MS (Method 1): Retention time=2.65 min. MS (ESI) m/z 618.2 [M+H]⁺.

Step 9. Synthesis of 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]benzoic Acid

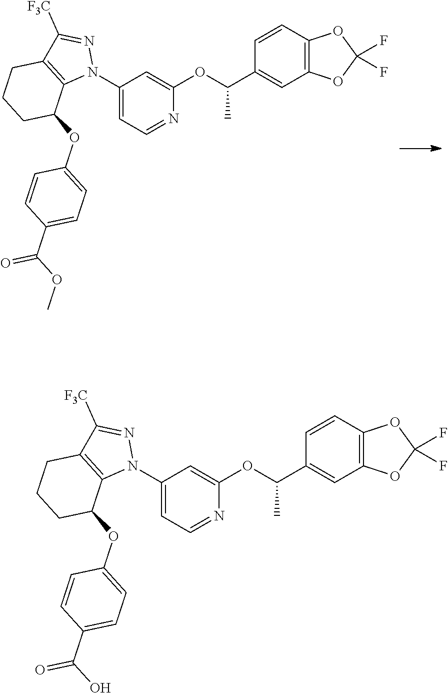 [SMILES: COC(=O)c1ccc(O[C@H]2CCCc3c(C(F)(F)F)nn(-c4ccnc(O[C@@H](C)c5ccc6c(c5)OC(F)(F)O6)c4)c32)cc1 ; C[C@H](Oc1cc(-n2nc(C(F)(F)F)c3c2[C@@H](Oc2ccc(C(=O)O)cc2)CCC3)ccn1)c1ccc2c(c1)OC(F)(F)O2]

To the solution of methyl 4-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7- yl)oxy)benzoate (product P1 from Step 8 above) (300 mg, 0.486 mmol) in methanol (10 mL), THF (10 mL) and water (5 mL) was added lithium hydroxide (47 mg, 1.94 mmol). The solution was then stirred at 25° C. overnight. The solution was concentrated under vacuum and purified by prep-HPLC to get the title compound (185 mg, 48% yield) as white solid. LC-MS (Method 1): Retention time=1.86 min. MS (ESI) m/z 602.0 [M-H]⁺. ¹H NMR (500 MHz, CH₃OH-d₄)δ8.06 (d, J=7.5 Hz, 1H), 8.03 (d, J=11 Hz, 2H), 7.16 (dd, J=7.0, 2.5 Hz, 1H), 7.12 (dd, J=7.0, 5.0 Hz, 2H), 7.05 (d, J=1.7 Hz, 1H), 7.03-6.87 (m, 3H), 6.03 (q, J=8.0 Hz, 1H), 5.74 (t, J=4.5 Hz, 1H), 2.87 (d, J=21 Hz, 1H), 2.63 (ddd, J=16.7, 11.0, 5.7 Hz, 1H), 2.29 (d, J=17 Hz, 1H), 2.00 (dd, J=12.2, 6.9 Hz, 1H), 1.95-1.75 (m, 2H), 1.43 (d, J=8.0 Hz, 3H).

### Synthetic Method 2 of the Synthesis of Example 3, Compound 3.

### Synthesis of 4-[[(7S)-1-[2-[(1S)-1-(2,2-difluoro-1,3-benzodioxol-5-yl)ethoxy]-4-pyridyl]-3-(trifluoromethyl)-4,5,6,7- tetrahydroindazol-7-yl]oxy]benzoic Acid

Step 1. Synthesis of 1-(2,2-difluorobenzo[d][1,3]dioxol-5-yl)ethan-1-one

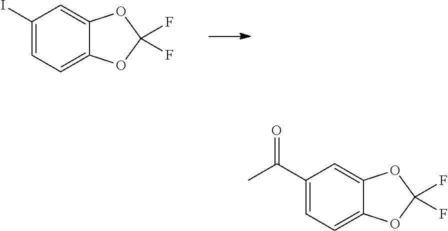

To a solution of 1-ethoxyvinyltri-n-butyltin (1.7 mL, 4.93 mmol) in toluene (20 mL) was added 2,2-difluoro-5-iodo-1,3-benzodioxole (1.40 g, 4.93 mmol) and Pd(PPh₃)₂Cl₂ (138 mg, 0.20 mmol). The reaction was stirred at 90° C. for 12 h under N₂, then cooled to rt. HCl (30 mL, 60.0 mmol) was added and stirred for 0.5 h. The mixture was extracted with EA (30 mL×3). The organic layers were combined, washed with brine (30 mL), dried over Na₂SO₄, filtered and concentrated. The crude product was purified by flash column chromatography eluted with 5% ethyl acetate in isohexane to get the title compound (950 mg, 95% yield) as a colorless oil. LC-MS (Method 1): Retention time=2.02 min. MS (ESI) m/z 201.0 (M+H)⁺. ¹H NMR (500 MHz, CDCl₃)δ7.77 (dd, J=8.3, 1.6 Hz, 1H), 7.69 (d, J=1.6 Hz, 1H), 7.14 (d, J=8.3 Hz, 1H), 2.60 (s, 3H).

Step 2. Synthesis of (R)-1-(2,2-difluorobenzo[d][1,3]dioxol-5-yl)ethan-1-ol

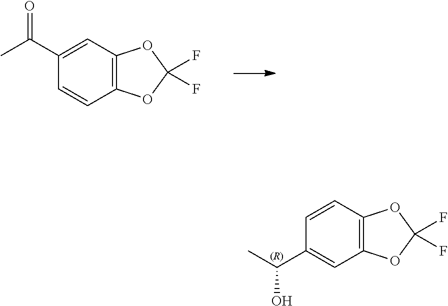

To a solution of(S)-2-methyl-CBSoxazaborolidine (Ref. 1) (8.25 g, 0.03 mol) in DCM (200 mL) under N₂ was added BH₃·Me₂S (150 mL, 2 M in DCM) at 0° C. The reaction was stirred at room temperature for 0.5 h, and then the solution of 1-(2,2-difluorobenzo[d][1,3]dioxol-5-yl)ethan-1-one (60 g, 0.30 mol) in DCM (300 mL) was added dropwise at 0° C. for 1 h. The reaction mixture was stirred at room temperature for 4 h. The reaction was quenched by addition of MeOH at 0° C., and then concentrated in vacuo to give the residue which was purified by flash silica gel column chromatography (ethyl acetate in petroleum ether from 0% to 10%) to afford the title compound (56 g, 92% yield) as a light-yellow liquid. LC-MS: Retention time=1.75 min. MS (ESI) m/z 185.0 [M-H₂O+H]⁺.

Step 3. Synthesis of 4-bromo-2-(1-phenylethoxy)pyridine

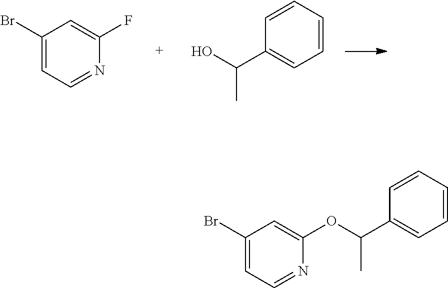

To a solution of 4-bromo-2-fluoro-pyridine (20.0 g, 114 mmol) and 1-phenylethanol (13.9 g, 114 mmol) in DMF (200 mL) was added Cs₂CO₃ (111 g, 342 mmol). The reaction was stirred at 100° C. overnight. The mixture was poured into water (2 L) and extracted with EA (200 mL×3). The organic layers were combined, washed with brine, dried over Na₂SO₄, filtered, and concentrated. The crude was then purified by flash column chromatography eluting with 10% ethyl acetate in PE to get the title compound (29.0 g, 91.8%) as a yellow solid. LCMS (Method 1): Retention time=2.45 min. MS (ESI) m/z 174.1, 176.1 [M-103]⁺.

Step 4. Synthesis of Tert-butyl 1-(2-(1-phenylethoxy)pyridin-4-yl)hydrazinecarboxylate

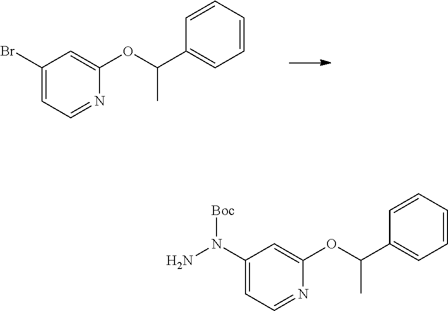 [SMILES: CC(Oc1cc(Br)ccn1)c1ccccc1 ; CC(Oc1cc(N(N)C(=O)OC(C)(C)C)ccn1)c1ccccc1]

To the solution of 4-bromo-2-(1-phenylethoxy)pyridine (15.0 g, 53.9 mmol) in 1,4-dioxane (150 mL) were added tert-butyl N-amino carbamate (8.6 g, 64.7 mmol), XantPhos (3.1 g, 5.39 mmol), tris(dibenzylideneacetone) dipalladium (4.9 g, 5.39 mmol), and Cs₂CO₃ (52.8 g, 162 mmol). Then the mixture was stirred at 100° C. overnight under Ar. The mixture was poured into water (2 L) and extracted with EA (200 mL×3). The organic layers were combined, washed with brine, dried over Na₂SO₄, filtered, and concentrated. The crude was then purified by flash column chromatography eluting with 10% ethyl acetate in PE to get the title compound (16.0 mg, 90.1%) as a yellow solid. LCMS (Method 1): Retention time=2.11 min. MS (ESI) m/z 226 [M-103]⁺.

Step 5. Synthesis of 1-(2-hydroxypyridin-4-yl)-3-(trifluoromethyl)-5,6-dihydro-1H-indazol-7(4H)-one

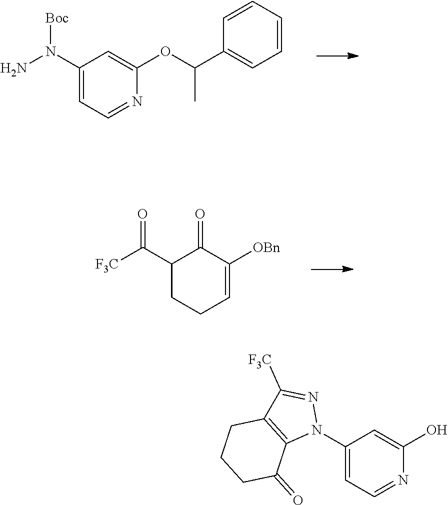 [SMILES: CC(Oc1cc(N(N)C(=O)OC(C)(C)C)ccn1)c1ccccc1 ; O=C1C(OCc2ccccc2)=CCCC1C(=O)C(F)(F)F ; O=C1CCCc2c(C(F)(F)F)nn(-c3ccnc(O)c3)c21]

To a solution of tert-butyl N-amino-N-[2-(1-phenylethoxy)-4-pyridyl]carbamate (10.0 g, 30.4 mmol) in 2,2,2-trifluoroethanol (100 mL) was added sulfuric acid (25 mL). Then the mixture was stirred at 80° C. overnight. The mixture was poured into water (500 mL) and adjusted pH to 8-9 with NaHCO₃, then extracted with EA (50 mL×3). The organic layers were combined, washed with brine, dried over Na₂SO₄, filtered, and concentrated. The crude was then purified by flash column chromatography eluting with 10% ethyl acetate in PE to get the title compound (5.0 g, 55.4%) as a yellow solid. LCMS (Method 1): Retention time=1.72 min. MS (ESI) m/z 298.3 [M+H]⁺.

Step 6. Synthesis of (S)-1-(2-(1-(2,2-difluorobenzo[d][1,3]dioxol-5-yl)ethoxy)pyridin-4-yl)-3-(trifluoromethyl)-5,6-dihydro-1H-indazol-7(4H)-one

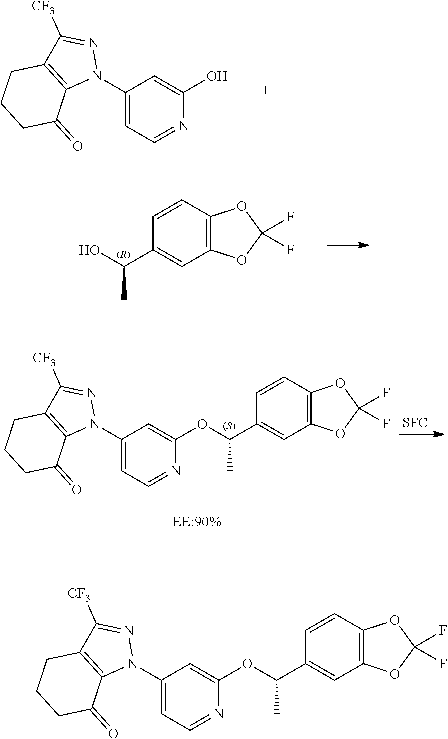

To a solution of 1-(2-hydroxy-4-pyridyl)-3-(trifluoromethyl)-5,6-dihydro-4H-indazol-7-one (4000 mg, 13.5 mmol) in THF (50 mL) were added (R)-1-(2,2-difluorobenzo[d][1,3]dioxol-5-yl)ethan-1-ol (Product from Step 2 above) (3265 mg, 16.1 mmol), and PPh₃ (4236 mg, 16.1 mmol). After the mixture was cooled to 0° C., DEAD (2.6 mL, 16.1 mmol) was added dropwise at 0° C. and then stirred at rt for 1 h under N₂. The mixture was concentrated, and the crude was then purified by flash column chromatography eluting with 10% ethyl acetate in PE to get the crude compound (4.5 g, ee 90%). This crude compound was purified further by SFC to afford the title compound (3.2 g, 49%, ee>99%) as a white solid. LCMS (Method 2): Retention time=2.36 min. MS (ESI) m/z 298.0 (M-183)⁺.

Step 7. Synthesis of (R)-1-(2-((S)-1-(2,2-difluorobenzo[d][1,3]dioxol-5-yl)ethoxy)pyridin-4-yl)-3-(trifluoromethyl)-4,5,6,7-tetrahydro-1H-indazol-7-ol

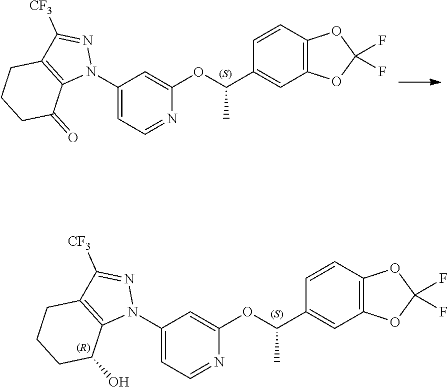

In a 200 mL reactor, the product from Step 6 (5.0 g, 10.4 mmol) was suspended in IPA (yellow suspension) (10 vol) at room temperature. TEA (0.6 vol) and catalyst (R, R)-TsDPEN-RuCl (p-cymene) (0.006 wt) were added to the mixture, and 3 cycles of vacuum/nitrogen exchange were applied. Formic acid (0.4 vol) was added slowly to the mixture in 10 min. (exothermic reaction from 20 to 25° C.). Temperature was increased to 45° C. and the solution was stirred for 4 h. Reaction mixture was concentrated under vacuum. Water (10 vol) was added and adjusted pH to 7-8 with 5% NaHCO₃ aqueous solution, then extracted with EA (2 vol×3). The organic layers were combined, washed with brine, dried over Na₂SO₄, filtered and concentrated. The crude was purified by flash column chromatography eluting with 20% ethyl acetate in isohexane to get the title compound (3.0 g, 60%) as a yellow solid. LCMS (Method 1): Retention time=1.73 min. MS (ESI) m/z 300 [M-183]⁺.

Step 8. Synthesis of Methyl 4-(((S)-1-(2-((S)-1-(2,2-difluorobenzo[d][1,3]dioxol-5-yl)ethoxy)pyridin-4-yl)-3-(trifluoromethyl)-4,5,6,7- tetrahydro-1H-indazol-7-yl)oxy)benzoate

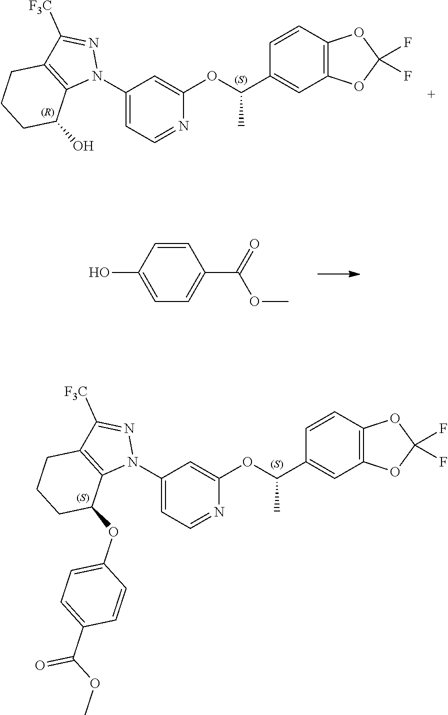

To a solution of (R)-1-(2-((S)-1-(2,2-difluorobenzo[d][1,3]dioxol-5-yl)ethoxy)pyridin-4-yl)-3-(trifluoromethyl)-4,5,6,7-tetrahydro-1H-indazol- 7-ol (product from Step 7 above) (3000 mg, 6.2 mmol) in THF (10 mL) were added methyl 4-hydroxybenzoate (1.1 g, 7.4 mmol) and PPh₃ (2.4 g, 9.3 mmol). The mixture was cooled to 0° C. and DEAD (1.6 g, 9.3 mmol) was added dropwise at 0° C. under N₂. Then the mixture was stirred at rt for 1 h. The mixture was concentrated, and the crude was then purified by flash column chromatography eluting with 10% ethyl acetate in PE to get the title compound (3.0 g, 79%) as a white solid. LCMS (Method 1): Retention time=2.65 min. MS (ESI) m/z 434.2 [M-183]⁺.

Step 9. Synthesis of 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]benzoic Acid

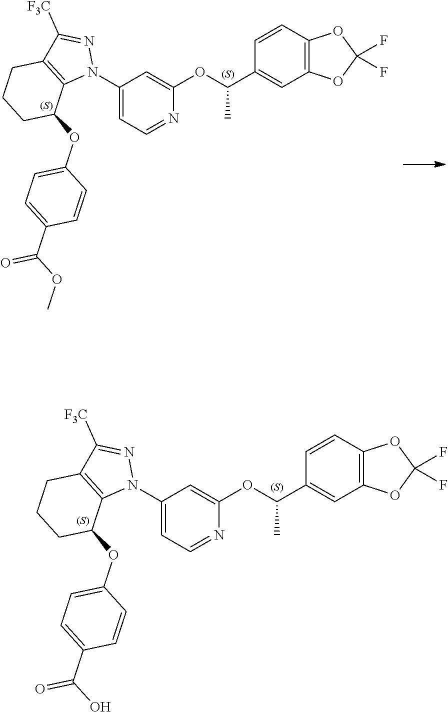

To a solution of methyl 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7- yl]oxy]benzoate (3.0 g, 4.86 mmol) in methanol (20 mL), THF (20 mL) and water (10 mL) was added lithium hydroxide (465 mg, 19.4 mmol). The reaction was stirred at 25° C. overnight. Then the mixture was concentrated, and the crude was purified by pre-HPLC (NH₄HCO₃) to get the tide compound (2204 mg, 75.2%) as a white solid.

The analytics (LCMS and ¹HNMR) are identical as the sample obtained through the synthetic Method 1.

### Example 4. Analysis of CFTR Maturation by Western Blot

### Cystic Fibrosis Submucosal Gland Epithelial Cells

CF submucosal gland epithelial cells (CFSMEo⁻) were derived from the airways of a CF patient (ΔF508/Q2X) and provided by Dr. Dieter Gruenert, University of California-San Francisco. CFSMEo⁻ cells were cultured at 37° C. with 5% CO₂ in minimum essential medium (MEM) with Earle's salt and nonessential amino acids, supplemented with 10% (volume per volume [v/v]) fetal bovine serum, 2 mM L-glutamine and 1× (v/v) penicillin/streptomycin and grown in tissue culture-treated flasks coated with an extracellular matrix (ECM) cocktail consisting of 10 μg/mL human fibronectin, 30 μg/mL bovine collagen type I, and 100 μg/mL bovine serum albumin in LHC basal medium.

### CFTR Mammalian Expression Constructs

Using standard techniques, CFTR and ΔF508-CFTR were each cloned into pcDNA3.0, featuring a cytomegalovirus promoter and SV40 early polyadenylation signal, for expression in mammalian cells (Sambrook et al, 1989).

### Nucleofection

CFSMEo⁻ cells were transiently transfected with CFTR expression constructs using the Lonza 4D-Nucleofector Core unit (Lonza, Catalogue No. AAF-1002B) with the X unit (Lonza, Catalogue No. AAF-1002X) and SF Cell Line 4D-Nucleofector X Kit L (Lonza, Lonza, Catalogue No. V4XC-2024) according to the manufacturer's instructions. In brief, cells were nucleofected with 4-μg plasmid deoxyribonucleic acid (DNA) at a concentration of 5.0×10⁷ cells/mL and plated at 2.4×10⁵ cells/well onto collagen-coated, 6-well plates (Corning Inc., Corning, NY).

### Western Blotting Assay

Transiently transfected CFSMEo⁻ cells expressing either wild-type CFTR or ΔF508-CFTR were plated at 3.2×10⁵ cells/well onto collagen-coated, 6-well plates (Corning Inc.). Twenty-four hours post-transfection, CFTR modulators were applied (3 wells per concentration) and allowed to incubate for 48 hours. Cell lysates were then harvested in cold IP lysis buffer (Pierce, Rockford, IL) supplemented with ethylenediaminetetraacetic acid (EDTA)-free protease inhibitors (Pierce). Following quantitation with a bicinchoninic acid assay (Pierce), 5 μg of each sample were separated on a Novex 4% to 12% Tris Glycine Plus gel (Invitrogen, Waltham, MA) and transferred onto a nitrocellulose membrane using the iBlot 2 Dry Blotting System (Invitrogen). Blots were then incubated with the UNC-596 human CFTR-specific monoclonal antibody (lot #596TJ100285) supplied by the Cystic Fibrosis Foundation, University of North Carolina-Chapel Hill. To control for loading, a second blot was incubated with the murine Na⁺/K⁺ATPase monoclonal antibody (Millipore, Catalogue No. 05-0369). Following normalization of each sample to the Na⁺/K⁺ATPase loading control, CFTR maturation levels were quantitated by densitometry using ImageJ software (National Institute of Health, Bethesda, MD).

### Example 5. Analysis of CFTR-HRP Trafficking

### Cystic Fibrosis Submucosal Gland Epithelial Cells

CF submucosal gland epithelial cells (CFSMEo⁻) were derived from the airways of a CF patient (ΔF508/Q2X) and provided by Dr. Dieter Gruenert, University of California-San Francisco. CFSMEo⁻ cells were cultured at 37° C. with 5% CO₂ in minimum essential medium (MEM) with Earle's salt and nonessential amino acids, supplemented with 10% (volume per volume [v/v]) fetal bovine serum, 2 mM L-glutamine and 1× (v/v) penicillin/streptomycin and grown in tissue culture-treated flasks coated with an extracellular matrix (ECM) cocktail consisting of 10 μg/mL human fibronectin, 30 μg/mL bovine collagen type I, and 100 μg/mL bovine serum albumin in LHC basal medium.

### CFTR Mammalian Expression Constructs

Using standard techniques, CFTR and ΔF508-CFTR were each cloned into pcDNA3.0, featuring a cytomegalovirus promoter and SV40 early polyadenylation signal, for expression in mammalian cells (Sambrook et al, 1989). For the HRP trafficking constructs, a 1059 bp CFTR fragment with an HRP tag inserted between residues S902 and Y903 of the 4th extracellular loop was synthesized by Genscript (Piscataway, NJ) and cloned into both CFTR and ΔF508-CFTR via flanking PmlI sites resulting in the pCMV-CFTR-HRP and pCMV-ΔF508-CFTR-HRP trafficking constructs.

### Nucleofection

CFSMEo⁻ cells were transiently transfected with CFTR expression constructs using the Lonza 4D-Nucleofector Core unit (Lonza, Catalogue No. AAF-1002B) with the X unit (Lonza, Catalogue No. AAF-1002X) and SF Cell Line 4D-Nucleofector X Kit L (Lonza, Lonza, Catalogue No. V4XC-2024) according to the manufacturer's instructions. In brief, cells were nucleofected with 4-μg plasmid deoxyribonucleic acid (DNA) at a concentration of 5.0×10⁷ cells/mL and plated at 2.4×10⁵ cells/well onto collagen-coated, 6-well plates (Corning Inc., Corning, NY).

### Horseradish Peroxidase Trafficking Assay

Transiently-transfected CFSMEo− cells were plated at 1.8×10⁴ cells/well onto collagen-coated, 96-well plates (Corning Inc.). Twenty-four hours post-transfection with either the wild-type CFTR HRP trafficking reporter pCMV-CFTR-HRP or with the ΔF508-CFTR HRP trafficking reporter pCMV-ΔF508-CFTR-HRP, CFTR modulators were applied (3 wells per concentration) and allowed to incubate for 48 hours. To detect the extracellular HRP tag fused to CFTR, cell culture media was removed, cells were washed with PBS and 100 μL of cell impermeable Luminata Forte HRP chemiluminescent substrate (EMD Millipore, Burlington, MA) was added to each well. Following a 5-minute incubation, chemiluminescence was measured using an En Vision plate reader (Perkin Elmer, Waltham, MA).

### Example 6. Analysis of CFTR Channel Function

### Study Design

CFhBE cells or CFTR wild-type hBE cells were expanded and seeded onto a semipermeable membrane and cultured for 15 to 20 days at an air-liquid interface to allow for the formation of tight junctions and apically-directed CFTR expression. At this time, CFhBE were treated with the indicated modulators using a Tecan D300 digital dispenser and allowed to incubate for 48 hours at 37° C. in a humidified chamber. After 48 hours cells were loaded onto TECC24 platforms (EP Design, Bertem, Belgium) in horizontal orientation and plates were filled with symmetric physiologic saline with glucose (“PS+”, in mM: 150 NaCl, 5 KCl, 2 CaCl, 1 MgCl, 10 HEPES, 10 glucose, pH 7.4 with HCl). Transepithelial voltage and resistance were recorded in current clamp mode, and the equivalent current (Ieq) was calculated according to Ohm's Law (V=IR, where V is the voltage across the conductor, I is the current flowing through the conductor, and R is the resistance provided by the conductor to the flow of current). After obtaining a baseline current reading, 10 μM benzamil was added to inhibit the epithelial sodium channel and thus eliminate all non-CFTR conductance. Once a stable baseline had been achieved, forskolin (10 μM) was then added to stimulate CFTR-dependent chloride conductance; finally, 20 μM bumetanide was added to inhibit chloride transport.

### Data Analysis

Bar graphs were plotted by subtracting the baseline minimum current after benzamil addition, and the area under the curve (AUC) between forskolin and bumetanide addition was calculated for each well and values are expressed as either fold over tezacaftor/ivacaftor/elexacaftor alone, which was used as the in-plate control, or as a percent of wild type CFTR current. The AUC was used as the measure of CFTR activity and functional restoration of epithelial chloride secretion in response to different therapeutic agents. Bar graphs are displayed as mean±standard error (SE).

1. A method of treating CFTR-mediated disease or disorder in a subject in need thereof, comprising administering to the subject a therapeutically effective amount of an NBD1 corrector in combination with an ICL4 corrector.

2. The method of claim 1, wherein the NBD1 corrector and the ICL4 corrector are administered simultaneously.

3. The method of claim 1, wherein the NBD1 corrector is administered prior to the ICL4 corrector.

4. The method of claim 1, wherein the NBD1 corrector is administered after the ICL4 corrector.

5. A method of treating CFTR-mediated disease or disorder in a subject in need thereof, comprising administered to the subject a therapeutically effective amount of an NBD1 corrector, wherein the subject has previously received treatment with an ICL4 corrector.

6. A method of treating CFTR-mediated disease or disorder in a subject in need thereof, comprising administered to the subject a therapeutically effective amount of an ICL4 corrector, wherein the subject has previously received treatment with an NBD1 corrector.

7. The method of any of claims 1-6, further comprising administering to the subject a TMD1 corrector.

8. A method of treating CFTR-mediated disease or disorder in a subject in need thereof, comprising administering to the subject a therapeutically effective amount of an NBD1 corrector in combination with an TMD1 corrector.

9. The method of claim 1, wherein the NBD1 corrector and the TMD1 corrector are administered simultaneously.

10. The method of claim 1, wherein the NBD1 corrector is administered prior to the TMD1 corrector.

11. The method of claim 1, wherein the NBD1 corrector is administered after the TMD1 corrector.

12. A method of treating CFTR-mediated disease or disorder in a subject in need thereof, comprising administered to the subject a therapeutically effective amount of an NBD1 corrector, wherein the subject has previously received treatment with an TMD1 corrector.

13. A method of treating CFTR-mediated disease or disorder in a subject in need thereof, comprising administered to the subject a therapeutically effective amount of an TMD1 corrector, wherein the subject has previously received treatment with an NBD1 corrector.

14. The method of any of claims 8-13, further comprising administering to the subject a ICL4 corrector.

15. A method of treating CFTR-mediated disease or disorder in a subject in need thereof, comprising administering to the subject a therapeutically effective amount of an NBD1 corrector in combination with a CFTR potentiator.

16. The method of claim 1, wherein the NBD1 corrector and the CFTR potentiator are administered simultaneously.

17. The method of claim 1, wherein the NBD1 corrector is administered prior to the CFTR potentiator.

18. The method of claim 1, wherein the NBD1 corrector is administered after the CFTR potentiator.

19. A method of treating CFTR-mediated disease or disorder in a subject in need thereof, comprising administered to the subject a therapeutically effective amount of an NBD1 corrector, wherein the subject has previously received treatment with a CFTR potentiator.

20. A method of treating CFTR-mediated disease or disorder in a subject in need thereof, comprising administered to the subject a therapeutically effective amount of a CFTR potentiator, wherein the subject has previously received treatment with an NBD1 corrector.

21. The method of any of claims 15-20, further comprising administering to the subject a TMD1 corrector.

22. The method of any of claims 15-21, further comprising administering to the subject an ICL4 corrector.

23. The method of any of claims 1-22, wherein the NBD1 corrector is a compound represented by 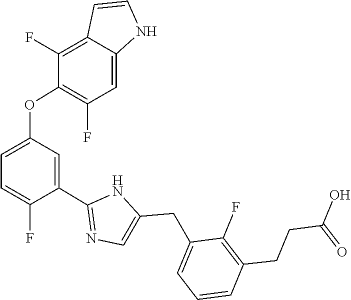 [SMILES: O=C(O)CCc1cccc(Cc2cnc(-c3cc(Oc4c(F)cc5[nH]ccc5c4F)ccc3F)[nH]2)c1F]
or a pharmaceutically acceptable salt thereof.

24. The method of any of claims 1-23, wherein the ICL4 corrector is a compound represented by 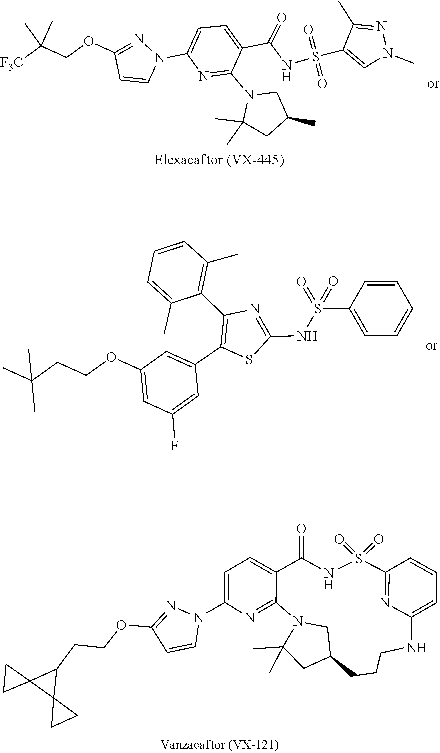
or a pharmaceutically acceptable salt thereof.

25. The method of any of claims 1-23, wherein the TMD1 corrector is a compound selected from the group consisting of 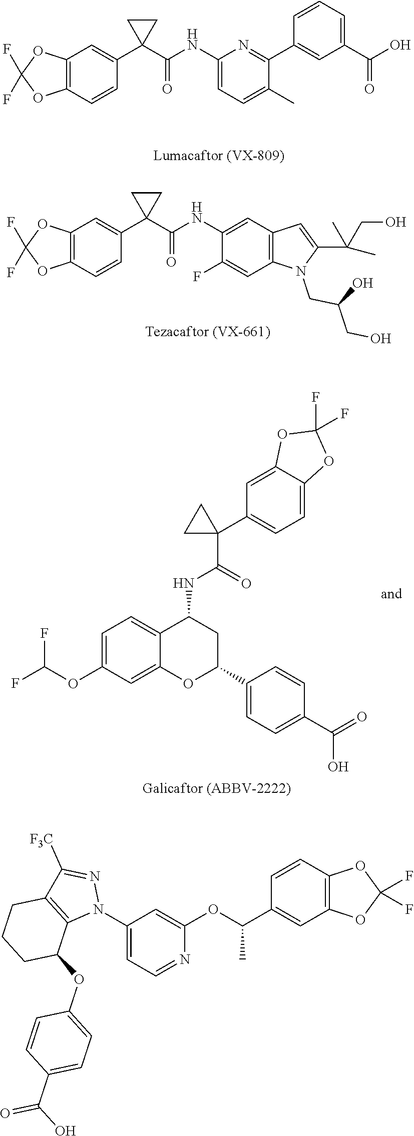 [SMILES: Cc1ccc(NC(=O)C2(c3ccc4c(c3)OC(F)(F)O4)CC2)nc1-c1cccc(C(=O)O)c1 ; CC(C)(CO)c1cc2cc(NC(=O)C3(c4ccc5c(c4)OC(F)(F)O5)CC3)c(F)cc2n1C[C@@H](O)CO ; O=C(O)c1ccc([C@H]2C[C@@H](NC(=O)C3(c4ccc5c(c4)OC(F)(F)O5)CC3)c3ccc(OC(F)F)cc3O2)cc1 ; C[C@H](Oc1cc(-n2nc(C(F)(F)F)c3c2[C@@H](Oc2ccc(C(=O)O)cc2)CCC3)ccn1)c1ccc2c(c1)OC(F)(F)O2]
or a pharmaceutically acceptable salt thereof.

26. The method of any of claims 1-25, wherein the CFTR potentiator is a compound represented by 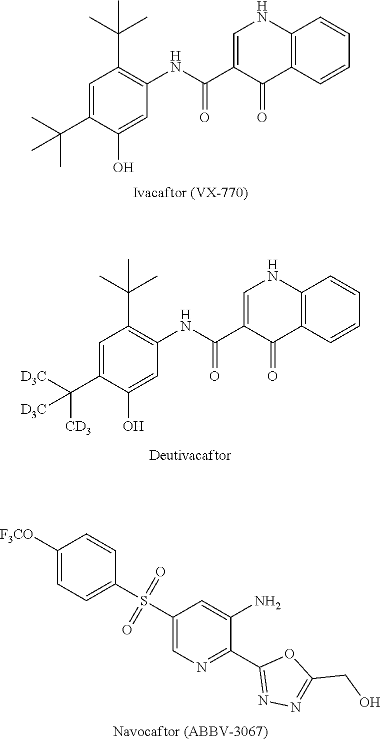 [SMILES: CC(C)(C)c1cc(C(C)(C)C)c(NC(=O)c2c[nH]c3ccccc3c2=O)cc1O ; [2H]C([2H])([2H])C(c1cc(C(C)(C)C)c(NC(=O)c2c[nH]c3ccccc3c2=O)cc1O)(C([2H])([2H])[2H])C([2H])([2H])[2H] ; Nc1cc(S(=O)(=O)c2ccc(OC(F)(F)F)cc2)cnc1-c1nnc(CO)o1]
or a pharmaceutically acceptable salt thereof.

27. The method of any of claims 1-26, wherein the CFTR-mediated disease or disorder is selected from the group consisting of cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

28. The method of claim 27, wherein the CFTR-mediated disease or disorder is selected from the group consisting of disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

29. The method of any of claims 1-28, wherein the CFTR-mediated disease or disorder is cystic fibrosis.

30. The method of any of claims 1-29, wherein the subject is human.
